(19) United States
(12) Patent Application Publication
(10) Pub. No.: US 2006/0078589 A1
(43) Pub. Date: Apr. 13, 2006

(54) Device for treating oral wound gaps

(76) Inventors: Steven Dee Jensen, South Jordan, UT (US); Shaneen Wintch, Kearns, UT (US)

Correspondence Address: Steven Jensen, 1190 W. Chavez Dr., So. Jordan, UT, 84095, US

(21) Appl. No.: 10/961,604

(22) Filed: Oct. 12, 2004

(51) Int. Cl. A61K 31/717; A61F 2/00

(52) U.S. Cl. 424/426; 514/57

(57) ABSTRACT

A device for treating oral wounds that form a gap and hence too large to suture. The device is intended to fill the resulting wound gap and upon contact with bleeding tissues cause local hemostasis. The device will remain in and protect the wound gap during the healing process. The coagulum plug that is created by this device will eventually heal over as permanent soft tissue and the embedded device removed by the physiological processes of the body. The device is in the shape of a compressible pellet that is packed in the wound gap to help increase retention by exerting an outward pressure. An embodiment of this device is an oxidized cellulose pellet.

### CROSS REFERENCE TO RELATED APPLICATIONS

U.S. Patent Documents

U.S. Pat. No. 4,597,960 July 1986 Cohen
U.S. Pat. No. 5,414,079 May 1995 Banker et al..
U.S. Pat. No. 6,536,448 March 2003 McDevitt, et al..
U.S. Pat. No. 6,500,777 December 2002 Wiseman, et al..
U.S. Pat. No. 6,306,154 October 2001 Hudson, et al..
U.S. Pat. No. 6,162,241 December 2000 Coury, et al..
U.S. Pat. No. 4,372,314 February 1983 Wall
   Other Publications
The Merk Index Thirteenth Edition, 2001—page 1242

### STATEMENT REGARDING FEDERALLY SPONSORED RESEARCH AND DEVELOPMENT

Not applicable.

### REFERENCE TO SEQUENCE LISTING, A TABLE, OR A COMPUTER PROGRAM LISTING COMPACT DISK APPENDIX

Not applicable.

### BACKGROUND OF THE INVENTION

Dental practitioners often encounter patients with open wounds, these can be caused by accidents or through routine surgery. The principal method of treating these wounds is suturing the wound together. There are some wounds that result in a gap or void in soft tissues, in these cases suturing is not feasible or practical. For instance, during a tooth extraction a large bleeding gap or socket is created, this gap isn't feasible to suture because there is not two soft tissue surfaces to unite as a whole.

The bodies method of repairing open wounds is to fill them with blood that eventually coagulates to form a soft plug, and if left undisturbed will eventually heal. This coagulum plug is a barrier of cells that inhibit the ingress of bacteria, thus preventing infection. This plug is also vital in the process of cell replacement during the formation of new soft tissue. If the soft plug were to be dislodged before it fully healed it creates a problem known to dentists as “dry sockets”. Where the bodies original intent was to repair the wound socket with new soft tissue, a dry socket is a socket from which the coagulum plug has been dislodged and the resulting hole eventually heals over. This soft coagulum plug is a plague to dental practitioners. This soft plug is easily removed by ordinary events that occur in the mouth everyday. Events as small as eating or sucking on a straw will dislodge this soft coagulum plug.

Open wound gaps create a variety of problems in the oral environment. A bleeding gap left unfilled is an ideal place for the compaction of food while eating. In the bacteria filled oral cavity, these food filled gaps become breeding grounds for infections. Therefore the central treatment has been to council patients to keep this area clean without disturbing this newly formed coagulated plug. Dental practitioners to encourage their patients to keep these areas clean even provide squirt bottles as a practical means of removing any debris. The degree of success in this healing regime is entirely dependent on patient compliance. With great difficulty, patients must execute constant vigilance in order to avoid dislodging the newly formed coagulum plug. Patients must also routinely clean these healing areas or likely suffer from infections. This is due to the soft delicate nature of a newly formed coagulum plug. Even patients who perform perfect cleaning regimes are still prone to infections.

The medical company Upjohn® markets a “sterile absorbable gelatin sponge” called Gel Foam®, this product comes in flat sheets. This product when placed onto bleeding tissues is intended to absorb blood like a sponge and form a coagulum. This product has the added advantage of being physiologically absorbed by the body in the event the material becomes trapped inside healing tissues. The disadvantage of Gel Foam® is that it was not designed to withstand the oral environment. Gel Foam® is made from gelatin, a digestible food. Once placed into the oral environment it begins to be broken down by saliva like any other food. The biggest disadvantage is the lack of physical cohesion within the material itself. Once the material contacts the fluids of bleeding tissues it converts to a slimy gel. This slimy gel acts almost as a lubricant on the surface of bleeding tissues. It also creates a delicate gel foam plug which is easily displaced by physical means. Gel Foam creates a soft coagulum plug that is easily removed by common events in the mouth like eating or brushing the teeth. In the oral environment, a Gel Foam® coagulated plug is not an ideal improvement over the bodies own healing process.

The medical field routinely uses oxidized cellulose in gauze form. Gauze is designed to treat surface wounds, not to fill in voids. A sheet of gauze is impractical when attempting to fill a wound gap because it must be methodically stuffed in bit by bit or rolled into a ball prior to being stuffed. A sheet of gauze would retain elastic memory and resist any attempt to be forced into a ball. A sheet of gauze forced into a ball would begin to open when the distorting force was removed. This distorting force would be cumbersome to dental practitioners because it requires them to stuff unruly edges of gauze where they need to be. A flat sheet is not ideal for packing a socket because of a loss of compressibility and control during placement.

What is needed is a device that can be placed to fill oral wound gaps with sufficient material cohesion and hemostatic properties, in order to create a more solid and retentive coagulum plug. This device must also remain during the healing cycle and be ultimately absorbed by physiological processes back into the tissues. It warrants that this device be in a form or shape that aids the practitioner in packing the wound gap, such as a compressible pellet.

### BRIEF SUMMARY OF THE INVENTION

Present invention provides a device for the treatment of oral wound gaps. The wound gap is packed with a material that creates local hemostasis on contact with bleeding tissues and aids in the formation of a solid and retentive coagulum plug. Said material must also remain in the coagulum plug throughout the healing process and eventually be removed by physiological processes back into the tissues. It warrants that this device be in a form or shape as to aid the practitioner in filling or packing the wound gap, such as a pellet. The preferred embodiment is an oxidized cellulose pellet.

### BRIEF DESCRIPTION OF SEVERAL VIEWS OF THE DRAWING

Not applicable.

### DETAILED DESCRIPTION OF THE INVENTION

Oxidized cellulose is commonly known as cellulosic acid, absorbable cellulose, or polyanhydroglucuronic acid. Oxidized cellulose is a chemically oxidized form of the common cellulose fiber such as cotton. Oxidized cellulose is cellulose that has increased its carboxylation content by varying the degree of oxidation. The degree of carboxylation can be estimated by the time it takes to dissolve oxidized cellulose in dilute alkaline solutions, such as 0.1-0.5 Molar sodium hydroxide. Since cellulose fibers are not soluble in dilute alkaline solutions and oxidized cellulose fibers are soluble.

The preferred method for manufacturing oxidized cellulose is the action of nitrogen dioxide gas on cellulose fiber. There are other oxidizing agents such as aqueous hypochlorite salts that will also create oxidized cellulose. Our experiments have shown that aqueous hypochlorite salts tend to degrade cellulose fibers. When cellulose fibers are placed in aqueous hypochlorite salts for more than one hour the fibers usually crumble apart, especially upon drying. We have found that one hour of reaction time does not create the degree of carboxylation necessary to impart adequate hemostatic properties to the fiber. Therefore the creation of oxidized cellulose by aqueous hypochlorite salts is impractical for our requirement of an intact oxidized fiber. We believe the degradation is due to the alkalinity of the hypochlorite solutions rather than to the oxidation process by the hypochlorite ion.

We have found that the action of nitrogen dioxide on cellulose fibers does not significantly degrade the strength of the fiber during the oxidation process. Cellulose fibers remain intact and do not crumble even after 48 hour exposure to nitrogen dioxide. The preferred process to create nitrogen dioxide gas is the action of manganese dioxide or disulfide on concentrated nitric acid. The action of manganese dioxide or disulfide on nitric acid is catalytic, any amount of nitrogen dioxide can be created by the metered addition of nitric acid to this catalyst. During this reaction there is also the significant formation of dinitrogen tetroxide which does not interfere in the oxidation process. We have found that the action of formaldehyde on concentrated nitric acid is another method for creating nitrogen dioxide. Formaldehyde is not the preferred method since the reaction is not catalytic and is consumed in the reaction.

The preferred method of manufacture is to take unaltered cellulose fibers and introduce them into a reaction vessel, while in another enclosed vessel concentrated nitric acid is metered into manganese dioxide powder. The nitrogen dioxide gas that is evolved is then piped to the vessel containing the unaltered cellulose. This vessel is then purged entirely with excess amounts of nitrogen dioxide and left sealed for 36 hours. The oxidized cellulose is then removed and washed in dilute sodium bicarbonate solution, followed by multiple agitated rinses in distilled water. The resulting oxidized cellulose is sufficiently carboxylated to create rapid local hemostasis. The resulting fibers can also be autoclaved before patient use in the case the fibers become contaminated by bacteria.

The clinical indications of oxidized cellulose are maximized in the form or shape of a pellet. A dental practitioner appreciates devices that improve the efficacy and ease of use of any treatment. Oxidized cellulose is observed to be most useful for filling wound gaps when it is compressed into a pellet. A pellet made of loose fibers is compressible and therefore packable into a socket. This is ideal when attempting to fill a wound gap and desire it to remain throughout the entire healing regime.

The medical field routinely uses oxidized cellulose in gauze form. Gauze is designed to dress surface wounds, not to fill in voids. A sheet of gauze is impractical when attempting to fill a wound gap because it must be methodically stuffed in bit by bit, or rolled into a ball prior to being stuffed. A sheet of gauze would retain elastic memory and resist any attempt to be forced into a ball. A sheet of gauze forced into a ball would begin to open when the distorting force was removed. This distorting force would be cumbersome to dental practitioners because it requires them to stuff unruly edges of gauze where they need to be. A sheet of gauze also introduces unnecessary air pockets during packing because of a lack of uniformity when folding a sheet into a ball prior to placement or while randomly packing a flat sheet directly into a socket. It is clinically superior to have the oxidized cellulose in the most practical shape prior to use, rather than physically modifying an impractical form into a desired shape. A flat sheet is not ideal for packing a socket because of a significant lack of compressibility and control during placement. The difficulty of uniformly compressing sheets of gauze into wound gaps compromise the retention of the coagulum plug. The superior compressibility of a pellet is the vital factor in retaining the coagulum plug throughout the duration of the healing process. The ability of a pellet to pack into a wound gap and exert an outward retentive force is vital to the success of the treatment. Oxidized cellulose pellets packed into a wound gap will immediately control local bleeding and form a solid coagulum plug with retention that is superior to current treatments. The solid coagulum plug formed with packed oxidized cellulose pellets will remain during the healing cycle and eventually be absorbed by physiological processes.

There are multiple clinical applications for this oxidized cellulose pellet device. It is indicated for treating tooth extraction sockets, periodontal surgery, and apilectomy cases. It is especially indicated for patients who have a tendency to profusely bleed such as hemophiliac patients or patients on blood thinning medication. The many uses of this device will become apparent during routine use by dental practitioners.

We claim:

1. A device for treating oral wound gaps which is comprised of:
a. A material that fills in a gap produced by an oral wound.
b. A material that upon contact with bleeding tissues will create local hemostasis.
c. A material that remains in the wound gap during the healing process and is eventually absorbed by the physiological processes of the body
d. A compressible pellet.

2. A device as in claim 1 wherein it is comprised of an oxidized cellulose pellet.
